(19) United States
(12) Patent Application Publication
(10) Pub. No.: US 2024/0344110 A1
(43) Pub. Date: Oct. 17, 2024

(54) COMPOSITION FOR CELL LYSIS AND NUCLEIC ACID EXTRACTION, NUCLEIC ACID EXTRACTION METHOD USING SAME, AND MOLECULAR DIAGNOSTIC METHOD USING SAME

(71) Applicant: INVITROS CO., LTD., Seoul (KR)

(72) Inventors: Min Ji LIM, Daejeon (KR); Jan Di KIM, Bucheon-si Gyeonggi-do (KR); Se Ryun KIM, Gimpo-si, Gyeonggi-do (KR); Chang Ju PARK, Daejeon (KR)

(21) Appl. No.: 18/294,628

(22) PCT Filed: Aug. 1, 2022
(86) PCT No.: PCT/KR2022/011277

§ 371 Date: Feb. 2, 2024

(30) Foreign Application Priority Data
Aug. 2, 2021 (KR) 10-2021-0101536

(51) Int. Cl. C12Q 1/6806

(52) U.S. Cl. CPC C12Q 1/6806

(57) ABSTRACT

The present invention relates to a composition for cell lysis and nucleic acid extraction, a nucleic acid extraction method using the same, and a molecular diagnostic method using the same, and specifically, to a composition for cell lysis and nucleic acid extraction, a nucleic acid extraction method using the same, and a molecular diagnostic method using the same, wherein a composition containing an RNase inhibitor is used as a solution for nucleic acid extraction, and steps of heating to specific temperatures are included, whereby it is possible to minimize the time for molecular diagnosis by performing polymerase chain reaction without a separate elution process and purification process, and to reduce the cost of molecular diagnosis by minimizing the use of dedicated devices and consumables in extraction.

### TECHNICAL FIELD

This application claims the benefit of Korean Patent Application No. 10-2021-0101536, filed with the Korea Intellectual Property Office on Aug. 2, 2021, the entire content of which is included in the present invention.

The present invention relates to a composition for cell lysis and nucleic acid extraction, a nucleic acid extraction method using the same, and a molecular diagnostic method using the same, and specifically, to a composition for cell lysis and nucleic acid extraction, a nucleic acid extraction method using the same, and a molecular diagnostic method using the same, wherein a composition containing an RNase inhibitor is used as a solution for nucleic acid extraction, and steps of heating to specific temperatures are included, whereby it is possible to minimize the time for molecular diagnosis by performing a polymerase chain reaction without a separate elution process and purification process, and to reduce the cost of molecular diagnosis by minimizing the use of dedicated devices and consumables in extraction.

### BACKGROUND ART

Recently, as the causes of diseases have been interpreted at the genetic level based on the results of human genome research, the demand for manipulation and biochemical analysis of biological samples has gradually increased for the purpose of curing or preventing human diseases. In addition to disease diagnosis, technology for extracting and analyzing nucleic acids from biological samples or from samples containing cells has been required in various fields, including new drug development, preliminary testing for viral or bacterial infection, and forensic medicine.

Meanwhile, in order to perform molecular diagnosis, it is common to extract nucleic acid, which is DNA or RNA containing genetic information, from saliva or blood from a person infected with a virus or bacteria, and amplify the same to identify whether or not the person has been infected with the disease.

FIG. 1 is a flowchart showing a polymerase chain reaction according to a conventional art. Referring to FIG. 1, in the conventional art, it is common to extract (S30) nucleic acid containing genetic information from a sample (S10), amplify (S70 and S90) the nucleic acid by performing a polymerase chain reaction, and analyze the results. In order to extract the nucleic acid, lysis (S31), elution (S33), and purification (S50) processes are required. However, to extract the nucleic acid, the lysis and purification processes require dedicated extraction devices for nucleic acid extraction and articles (tools made of plastic, magnetic beads, solutions, etc.) that are consumed for extraction.

When nucleic acid is extracted using the above-described conventional art, the nucleic acid with high purity may be extracted, but a problem arises in that the extraction process takes a long time, and thus the conventional art is not suitable for diagnosis or medical examination in emergency situations or emergency rooms. In addition, when the conventional art is applied to a situation where national quarantine is necessary due to rapid virus spread, it is necessary to continuously use dedicated devices and consumables for extraction, and thus a problem arises in that high costs are incurred for diagnosis.

Therefore, in order to solve the above-described problems, there is an urgent need to develop a technology capable of performing a polymerase chain reaction only through cell lysis without an elution process or purification by using a specific composition.

### DISCLOSURE

### Technical Problem

An object of the present invention is to provide a composition for cell lysis and nucleic acid extraction and a molecular diagnostic method using the same, wherein a composition containing specific components is used in a process of lysing cells to extract nucleic acid from cells, a mixture containing the lysed cells is heated, thus omitting separate processes of purifying and eluting a solution containing the lysed cells, and a polymerase chain reaction may be performed using the solution containing the lysed cells.

However, objects to be achieved by the present invention are not limited to the object mentioned above, and other objects not mentioned above will be clearly understood by those skilled in the art from the following description.

### Technical Solution

One embodiment of the present invention provides a composition for cell lysis and nucleic acid extraction containing an RNase inhibitor and a buffer.

According to one embodiment of the present invention, the RNase inhibitor may include an RNase A inhibitor.

According to one embodiment of the present invention, the RNase inhibitor may be derived from protein.

According to one embodiment of the present invention, the buffer may have a pH of 6.0 to 9.0.

According to one embodiment of the present invention, the buffer may contain any one selected from among glycerol, hydroxyethyl piperazine ethane sulfonic acid (HEPES), dithiothreitol (DTT), potassium chloride, and combinations thereof.

One embodiment of the present invention provides a method for cell lysis and nucleic acid extraction including: a step of preparing a mixture by adding a sample containing nucleic acid to the composition for cell lysis and nucleic acid extraction; a first heating step of maintaining the mixture at a temperature of 25° C. to 45° C.; and a second heating step of maintaining the mixture resulting from the first heating step at a temperature of 75° C. or higher to lower than 100° C.

According to one embodiment of the present invention, the first heating step and the second heating step may each be performed for 1 minute to 30 minutes.

According to one embodiment of the present invention, the concentration of the RNase inhibitor in the mixture may be 7.5 units/reaction to 60.0 units/reaction, with respect to the volume of the mixture being 30 μL.

One embodiment of the present invention provides a molecular diagnostic method including: a step of adding a premix and a solution containing primers and a probe to a mixture containing the nucleic acid extracted by the method for cell lysis and nucleic acid extraction; and amplifying the extracted nucleic acid by polymerase chain reaction.

### Advantageous Effects

The composition for cell lysis and nucleic acid extraction according to one embodiment of the present invention may minimize the time required for molecular diagnosis by nucleic acid amplification by omitting separate elution and purification processes for a solution containing lysed cells and performing polymerase chain reaction using the solution containing lysed cells.

The method for cell lysis and nucleic acid extraction according to one embodiment of the present invention can improve the accuracy of molecular diagnosis by inactivating factors, which impede the accuracy of polymerase chain reaction, through heating in the process of extracting nucleic acid.

The molecular diagnostic method according to one embodiment of the present invention can reduce the cost of molecular diagnosis by minimizing the use of dedicated devices and consumables in extraction.

The effects of the present invention are not limited to the effects mentioned above, and effects not mentioned above will be clearly understood by those skilled in the art from the present specification and the accompanying drawings.

### BRIEF DESCRIPTION OF DRAWINGS

FIG. 1 is a flowchart showing a polymerase chain reaction according to a conventional art.

FIG. 2 shows a schematic diagram of a molecular diagnostic method according to one embodiment of the present invention and a schematic diagram showing the reaction of components inside a tube.

FIG. 3 is a flowchart of a molecular diagnostic method according to one embodiment of the present invention.

FIG. 4 shows a schematic diagram of molecular diagnostic methods according to Example 1 and a graph showing the Ct values measured in Examples 1-1 and 1-2.

FIG. 5 shows a schematic diagram of molecular diagnostic methods according to Example 2 and a graph showing the Ct values measured in Examples 2-1 to 2-4.

FIG. 6 shows a schematic diagram of molecular diagnostic methods according to Example 3 and a graph showing the Ct values measured in Examples 3-1 and 3-2.

FIG. 7 shows a schematic diagram of molecular diagnostic methods according to Example 4 and a graph showing the Ct values measured in Examples 4-1 and 4-2.

FIG. 8 is a schematic diagram showing molecular diagnostic methods according to Examples 4-3 to 4-6.

FIG. 9 is a graph showing the Ct value measured in a PCR including RNA extraction and purification processes according to a conventional art and the Ct value measured in a Preparation Example.

FIG. 10 is a graph showing a Ct value depending on the concentration of RNase inhibitor in a first heating step of a Preparation Example.

FIG. 11 is a graph showing a Ct value depending on the temperature of the first heating step of the Preparation Example.

### DESCRIPTION OF REFERENCE NUMERALS

S10: sample collection step; S30: extraction step
S31: lysis step; S33: elution step
S50: purification step; S70: RT-PCR
S90: PCR; S110: step of preparing sample-containing mixture
S130: first heating (incubation) step; S150: second heating (thermal lysis) step
S170: step of adding PCR sample; S190: step of amplifying nucleic acid
S191: RT-PCR; S193: PCR

### BEST MODE

Throughout the present specification, it is to be understood that when any part is referred to as “including” or “containing” any component, it does not exclude other components, but may further include other components, unless otherwise specified.

Throughout the present specification, “A and/or B” means “A and B” or “A or B”.

Hereinafter, the present invention will be described in more detail.

One embodiment of the present invention provides a composition for cell lysis and nucleic acid extraction containing an RNase inhibitor and a buffer.

The composition for cell lysis and nucleic acid extraction according to one embodiment of the present invention may minimize the time required for molecular diagnosis by nucleic acid amplification by omitting separate elution and purification processes for a solution containing lysed cells and performing a polymerase chain reaction using the solution containing lysed cells.

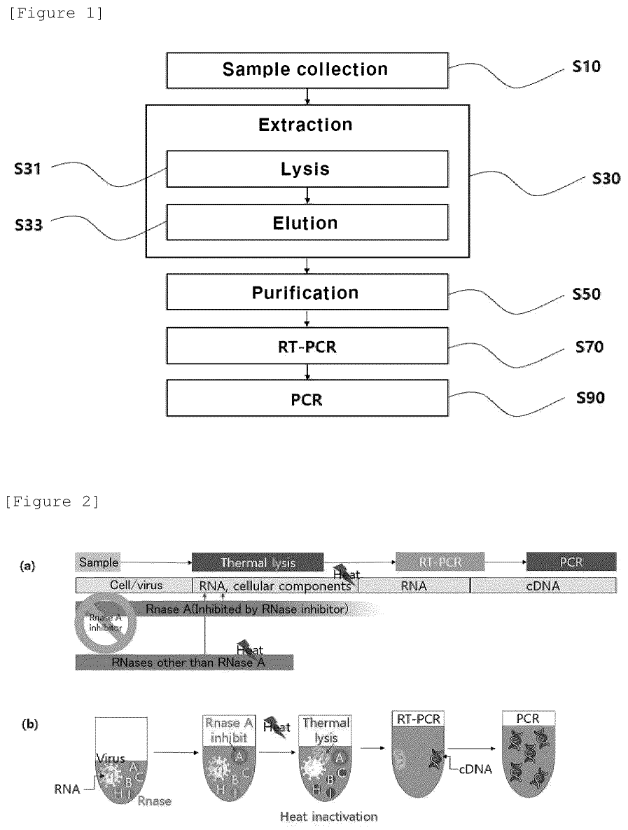

Referring to FIG. 1, in a molecular diagnostic method according to a conventional art, a sample is collected (S10), and then subjected to lysis (S31), elution (S33) and purification (S50) processes to extract a nucleic acid such as DNA or RNA from cells (S30), and an additional PCR buffer is added to the eluted solution, followed by reverse transcription PCR (RT-PCR, S70) and polymerase chain reaction (PCR, S90). Then, the nucleic acid amplified by the PCR is generally used for diagnosis. However, the above method has problems in that dedicated devices for the lysis, elution and purification processes are required, and in that various consumables such as solutions or plastic wares (plates and/or tubes) should be continuously used for these processes.

However, in the molecular diagnostic method according to the conventional art, the Ct value tends to be low because the sample is concentrated after extracting the nucleic acid and PCR is performed on the sample with high concentration. In contrast, in direct PCR (d-PCR), a cell sample is collected and diluted by adding a buffer for lysis, and thus the concentration of the sample to be subjected to PCR is lowered, and thus there is a limitation in that the Ct value is necessarily higher than that in the molecular diagnostic method according to the conventional art. Therefore, there has been a need for a molecular diagnostic method that can shorten the time by maximizing PCR efficiency by minimizing damage to and loss of RNA even when extracting the RNA from a small amount of a cell sample.

Furthermore, when a chemical lysis process is performed using a surfactant in conventional d-PCR, a problem arises in that RNase present together with the sampled cells is inevitably included. In other words, there are problems in that, in the process of sampling cells, RNase is accompanied with cells, and in the process of lysing the cells using a surfactant, the RNase degrades RNA from the cells, resulting in a rapid decrease in the efficiency of PCR.

According to one embodiment of the present invention, the composition for cell lysis and nucleic acid extraction includes an RNase inhibitor. Specifically, as the composition includes an RNase inhibitor that is capable of inhibiting RNase that is not inactivated even when heated, it may simplify molecular diagnosis and shorten the time required for molecular diagnosis.

According to one embodiment of the present invention, the RNase inhibitor may include an RNase A inhibitor. By selecting the RNase A inhibitor as the RNase inhibitor as described above, it is possible to inactivate theremostable RNase A while inhibiting other RNases by heating, thereby simplifying molecular diagnosis and shortening the time required for molecular diagnosis.

The characteristics of RNases are as shown in Table 1 below.

TABLE 1

| Type | RNase A | RNase T2 | RNase T1 | RNase H | RNase P | RNase I |
|---|---|---|---|---|---|---|
| Requirements | — | — | — | Divalent | Divalent | — |
| for RNA | | | | metal ion | metal ion | |
| cleavage | | | | | | |
| Principle of | Specifically | Cleave all | Cleave unpaired | Hydrolyze the RNA | Hydrolyze the | Similar to T2, |
| RNA cleavage | cleaves the 3′ | residues | G residues at 3′ | phosphodiester | phosphordiester | Cleave all |
| | end of unpaired | (preferring A | end | bond in | bond of pre-tRNA | residues |
| | cytosine/uracil | residue) | | DNA/RNA hybrid | | |
| | (pyrimidine) | | | | | |
| | residues in | | | | | |
| | ssRNA | | | | | |
| Temperature- | Stable at up to | — | 20° C. to 50° C. | Inactivated when | Active at 50° C. | Inactivated |
| related | 100° C. | | | heat-treated at | and inactivated | when heat- |
| properties | | | | 60° C. for 10 min | when heat-treated | treated at |
| | | | | | at 65° C. for 10 min | 70° C. for |
| | | | | | | 20 min |

Specifically, RNases correspond to enzymes that are affected by temperature. However, RNase T2, RNase T1, RNase H, RNase P, and RNase I, when heated, are inactivated at low temperatures and lose their RNA degradation activity, but RNase A is stable even when heated to 100° C. Thus, there is a problem in that it is not possible to inactivate all RNases by heating.

FIG. 2 shows a schematic diagram of a molecular diagnostic method according to one embodiment of the present invention and a schematic diagram showing the reaction of components inside a tube. Referring to FIGS. 2(a) and (b), a cell or virus sample is collected from the human body. The collected sample is added to the composition for cell lysis and nucleic acid extraction to prepare a mixture, and the RNase A inhibitor contained in the mixture inactivates RNase A contained in the sample. The mixture is heated to thermally inactivate all RNases other than RNase A, and at the same time, the cells are thermally lysed to extract nucleic acid, i.e., RNA or DNA, from the cells. When the extracted nucleic acid is mixed with primers, a probe and a premix and subjected to RT-PCR and PCR, the time for amplification of the nucleic acid may be shortened.

According to one embodiment of the present invention, the RNase inhibitor may be derived from protein. Specifically, the RNase inhibitor may have a large molecule size. That is, the RNase inhibitor may be derived from protein, may have a large molecule size, and may bind to RNase to form a large molecule, thereby preventing inhibition of PCR reaction. More specifically, the RNase inhibitor may be one selected from among one derived from murine lung, one derived from human placenta, or a combination thereof. The RNase inhibitor derived from murine lung may be Nanohelix RI (RNase Inhibitor). The RNase inhibitor may be one selected from the group consisting of Nanohelix HelixAyme RNase inhibitor (RNI2000), Themo Scientific RiboLock inhibitor (E00381), Invitrogen RNaseOUT recombinant ribonuclease inhibitor (10777019), Takara recombinant RNase inhibitor (2313A), Invitrogen™ SUPERase⋅In™ RNase inhibitor (AM2694), Applied Biosystems™ RNase inhibitor (N8080119), Roche Protector RNase inhibitor (RNAINH-RO/3335399001), Sigma-Aldrich ribonuclease inhibitor human (R2520), Promega RNasin@/RNasin® plus ribonuclease Inhibitor, NEW ENGLAND BioLabs Inc. RNase inhibitor, murine (M0314), NEW ENGLAND BioLabs Inc. RNase inhibitor, human placenta (M0307), ABclonal Technology RNase inhibitor, mammalian (RK21401), BioVision RNaseOFF ribonuclease inhibitor (M1238), PCR Biosystems RiboShield™ RNase inhibitor (PB30.23-02), Blirt RIBOPROTECT Hu RNase inhibitor (RT35), highQu GmbH SecurRIN™ Advanced RNase inhibitor (RNI0305), Enzynomics RNase inhibitor (M007), Meridian Bioscience RiboSafe RNase inhibitor (BIO-65027), QIAGEN RNase inhibitor (Y9240L), Lucigen RiboGuard™ RNase inhibitor (RG90925), Jena Bioscience RNase inhibitor-recombinant (PCR392S), abm RNaseOFF ribonuclease inhibitor (G138), biotechrabbit RNase inhibitor (BR0400901), BioFACT RNase inhibitor (RI 152-20h), Canvax RNase inhibitor (P0269), ShineGene RNasin (RNase inhibitor) (ZP00801), TOYOBO RNase inhibitor (SIN-201), or combinations thereof. As described above, the RNase inhibitor derived from protein is characterized by having a large molecule size. When the protein-derived RNase inhibitor with a large molecule size is used, it may prevent inhibition of polymerase chain reaction (PCR) in the molecular diagnosis process described later, but an RNase inhibitor derived from a chemical substance such as PVSA has a small molecular size, and thus may play a role in inhibiting polymerase chain reaction (PCR) in the molecular diagnosis process. It is known that chemically derived substances such as guanidinium isothiocyanate (GITC) also inhibit RNase, but contribute to inhibition of PCR. In addition, reducing agents such as beta-mercaptoethanol may also be used as RNase inhibitors, but reducing agents have disadvantages in terms of long-term storage and safety. Therefore, by selecting the RNase inhibitor from among those derived from proteins as described above, it is possible to shorten the time required for the molecular diagnostic process by preventing inhibition of polymerase chain reaction (PCR) in the molecular diagnostic process.

According to one embodiment of the present invention, the composition for cell lysis and nucleic acid extraction includes a buffer. As the composition for cell lysis and nucleic acid extraction includes a buffer as described above, it may reduce the time required for the molecular diagnostic process by improving the reactivity of polymerase chain reaction (PCR) in the molecular diagnostic process.

According to one embodiment of the present invention, the buffer may have a pH of 6.0 to 9.0. Specifically, the buffer may have a pH of 6.1 to 8.9, a pH of 6.2 to 8.7, a pH of 6.3 to 8.6, a pH of 6.4 to 8.5, a pH of 6.5 to 8.4, a pH of 6.6 to 8.3, a pH of 6.7 to 8.2, a pH of 6.8 to 8.1, a pH of 6.9 to 8.0, a pH of 7.0 to 7.9, a pH of 7.1 to 7.8, a pH of 7.2 to 7.7, a pH of 7.3 to 7.6, or a pH of 7.4 to 7.5. By controlling the pH of the buffer within the above-mentioned range, it is possible to improve the efficiency in the cell lysis process and promote the reaction between the RNase inhibitor and RNase.

According to one embodiment of the present invention, the buffer may include any one selected from the group consisting of glycerol, hydroxyethyl piperazine ethane sulfonic acid (HEPES), dithiothreitol (DTT), potassium chloride, and combinations thereof. As the buffer includes one selected from those described above, it may improve the efficiency in the cell lysis process, promote the reaction between the RNase inhibitor and RNase, and reduce the time required for the molecular diagnostic process by increasing the reactivity of polymerase chain reaction (PCR) in the molecular diagnostic process.

One embodiment of the present invention provides a method for cell lysis and nucleic acid extraction including: a step of preparing a mixture by adding a sample containing nucleic acid to the composition for cell lysis and nucleic acid extraction; a first heating step of maintaining the mixture at a temperature of 25° C. to 45° C.; and a second heating step of maintaining the mixture resulting from the first heating step at a temperature of 75° C. or higher to lower than 100° C.

The method for cell lysis and nucleic acid extraction according to one embodiment of the present invention can improve the accuracy of molecular diagnosis by inactivating factors, which impede the accuracy of polymerase chain reaction, through heating in the process of extracting nucleic acid.

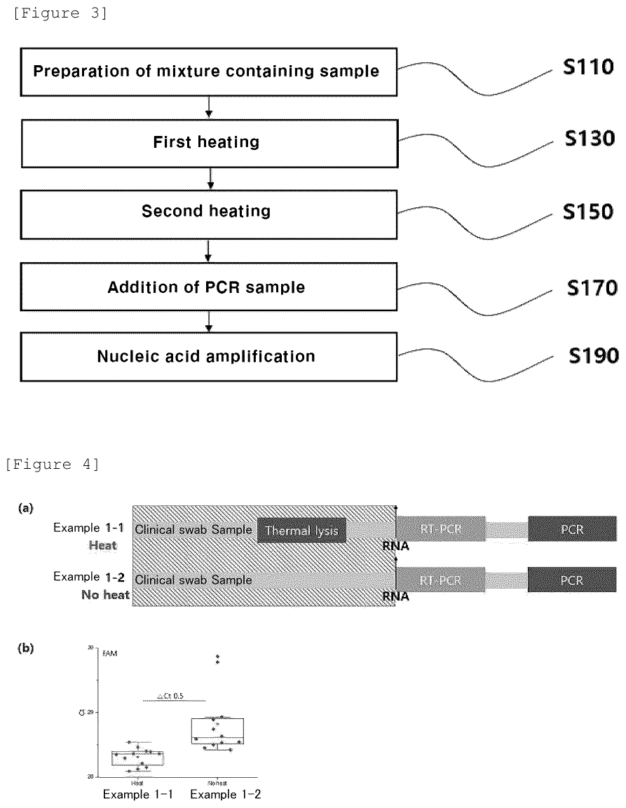

FIG. 3 is a flowchart of a molecular diagnostic method according to one embodiment of the present invention. The method may include: a sample collection step of collecting a biological sample from a human; a step (S110) of preparing a mixture by adding the collected sample to the composition for cell lysis and nucleic acid extraction; a first heating step (S130) of heating (incubating) the mixture; and a second heating step (S150) of heating (thermally lysing) the incubated mixture.

According to one embodiment of the present invention, the method includes a step (S110) of preparing a mixture by adding a sample containing nucleic acid to the composition for cell lysis and nucleic acid extraction. Specifically, since the mixture contains the composition for cell lysis and nucleic acid extraction and a sample containing nucleic acid, that is, a biological sample collected from a human and containing RNase, the RNase inhibitor contained in the composition may inactivate RNase, thereby protecting RNA and the like lysed out from the cells from RNase before performing the second heating (thermal lysis) step described later. Furthermore, by using a specific RNase inhibitor, it sensitivity by minimizing is possible to improve PCR unnecessary components and minimize the time required for molecular diagnosis.

In the method for cell lysis and nucleic acid extraction in the present specification, contents that overlap with those in the composition for cell lysis and nucleic acid extraction are omitted.

According to one embodiment of the present invention, the method may further include a step of collecting a biological sample from a human, before the step (S110) of preparing the mixture. Specifically, the biological sample collected from a human may be a cell sample containing nucleic acid, that is, DNA and/or RNA, such as blood, bodily fluid, saliva, etc., without being limited thereto. As the biological sample is collected from a human before the step (S110) of preparing the mixture as described above, it is possible to perform molecular diagnosis easily by amplifying a molecular diagnosis target, that is, the SAR-COV-2 gene, which is the cause of COVID 19.

According to one embodiment of the present invention, the method includes a first heating step (S130) of maintaining the mixture at a temperature of 25° C. to 45° C. Specifically, the first heating step, an incubation step, may be a step in which the RNase and the RNase inhibitor, which are contained in the mixture, are sufficiently combined with each other, thereby inactivating the RNase. More specifically, the temperature in the first heating step may be 26° C. to 44° C., 27° C. to 43° C., 28° C. to 42° C., 29° C. to 41° C., 30° C. to 40° C., 31° C. to 39° C., 32° C. to 38° C., 33° C. to 37° C., or 34° C. to 37° C. Most preferably, the temperature in the first heating step may be maintained at 37° C. By controlling the temperature in the first heating (incubation) step within the above-mentioned range, it is possible to inactivate the RNase by promoting the reaction between the RNase and the RNase inhibitor, and prevent degradation of RNA released from the cells by deactivating the RNase before thermal lysis of the cells.

According to one embodiment of the present invention, the method includes a second heating step (S150) of maintaining the mixture resulting from the first heating step at a temperature of 75° C. or higher to lower than 100° C. Specifically, the secondary heating step is a step of thermally lysing cells, which may be a step of lysing the cells in the sample contained in the mixture and containing nucleic acid, that is, the cell-containing biological sample collected from a human, thereby extracellularly exposing the DNA and/or RNA contained in the cells. Furthermore, thermal lysis of the cells and thermal inactivation that inactivates RNase may be simultaneously implemented, and additionally, inactivation of intracellular components that inhibit PCR may be simultaneously implemented. Specifically, the second heating step (S150) may simultaneously implement thermal lysis of cells, thermal inactivation of RNase, and inactivation of intracellular components. Specifically, the second heating step may be performed by maintaining the mixture from the first heating step at 76° C. to 99° C., 77° C. to 98° C., 76° C. to 97° C., 77° C. to 96° C., 78° C. to 95° C., 79° C. to 94° C., 80° C. to 93° C., 81° C. to 92° C., 82° C. to 91° C., 83° C. to 90° C., 84° C. to 89° C., 85° C. to 88° C., or 86° C. to 87° C. Preferably, the secondary heating step may be performed by maintaining the mixture resulting from the first heating step at 94.5° C. to 95.5° C., or 95° C. By controlling the temperature in the second heating (thermal lysis) step within the above-mentioned range, it is possible to eliminate the use of separate additives for inhibiting RNase, thereby maintaining the concentration of substances other than nucleic acid at a low level and excluding interference factors to the highest possible extent, and to prevent PCR from being inhibited due to separate additives for inhibiting RNase because the additives are not included. In addition, it is possible to inactivate RNases other than RNase A while inactivating intracellular substances, and expose the nucleic acid (DNA and/or RNA) within the cells by thermal lysis of the cells, thereby reducing the time required for molecular diagnosis.

According to one embodiment of the present invention, the method does not include a separate elution step and purification step, after extracting the nucleic acid from the sample containing nucleic acid. As the method does not include a separate elution step and purification step after nucleic acid extraction as described above, it is possible to reduce the time required for molecular diagnosis, and to reduce costs because consumables or dedicated devices for nucleic acid extraction are not used.

According to one embodiment of the present invention, the first heating step and the second heating step may each be performed for 1 minute to 30 minutes. Specifically, the first heating step and the second heating step may each be performed for 2 minutes to 29 minutes, 3 minutes to 28 minutes, 4 minutes to 27 minutes, 5 minutes to 26 minutes, 6 minutes to 25 minutes, 7 minutes to 24 minutes, 8 minutes to 23 minutes, 9 minutes to 22 minutes, 10 minutes to 21 minutes, 11 minutes to 20 minutes, 12 minutes to 19 minutes, 13 minutes to 18 minutes, 14 minutes to 17 minutes, or 15 minutes to 16 minutes. More specifically, the first heating step and the second heating step may each be performed for 4.5 minutes to 5.5 minutes, or 5 minutes. By controlling the time for which each of the first heating step and the second heating step is performed within the above-mentioned range, it is possible to maximize inactivation of RNase and improve the effect of thermal lysis of the cells.

According to one embodiment of the present invention, the concentration of the RNase inhibitor in the mixture may be 7.5 units/reaction to 60.0 units/reaction, with respect to the volume of the mixture being 30 μL. The concentration of the RNase inhibitor in the mixture may vary depending on an increase or decrease in the total volume of the mixture. Specifically, the concentration of the RNase inhibitor in the mixture may be 7.5 units/reaction to 60.0 units/reaction, 8.0 units/reaction to 59.0 units/reaction, 9.0 units/reaction to 58.0 units/reaction, 10.0 units/reaction to 57.0 units/reaction, 15.0 units/reaction to 55.0 units/reaction, 20.0 units/reaction to 50.0 units/reaction, 25.0 units/reaction to 45.0 units/reaction, or 30.0 units/reaction to 40.0 units/reaction. More specifically, the concentration of the RNase inhibitor in the mixture may be 7.5 units/reaction to 52.5 units/reaction, 30.0 units/reaction to 52.5 units/reaction, or 30.0 units/reaction to 45.0 units/reaction. By controlling the concentration of the RNase inhibitor in the mixture within the above-mentioned range, it is possible to maximize the inactivation of RNase before thermal lysis of the cells, and to prevent the RNA exposed from the cells from being degraded after thermal lysis of the cells, and also to reduce the time required for molecular diagnosis by minimizing factors that inhibit subsequent PCR.

In the present specification, “unit/reaction (U/rxn)” may refer to the amount of RNase inhibitor required to inhibit the activity of 5 ng RNase A by 50% per reaction.

One embodiment of the present invention provides a molecular diagnostic method including: a step of adding a premix and a solution containing primers and a probe to a mixture containing a nucleic acid extracted by the method for cell lysis and nucleic acid extraction; and a step of amplifying the extracted nucleic acid by polymerase chain reaction.

The molecular diagnostic method according to one embodiment of the present invention may reduce the cost of molecular diagnosis by minimizing the use of dedicated devices and consumables in extraction.

Referring to FIG. 3, according to one embodiment of the present invention, the method includes a step (S170) of adding a premix and a solution containing primers and a probe to a mixture containing a nucleic acid extracted by the method for cell lysis and nucleic acid extraction. In the present specification, a composition, containing a premix and a solution containing primers and a probe, may refer to a “PCR sample”. Specifically, as a PCR sample, containing a premix and a solution containing primers and a probe, is added to a mixture containing a nucleic acid extracted by the method for cell lysis and nucleic acid extraction, the components required for nucleic acid amplification may be complete and the nucleic acid may be easily amplified.

According to one embodiment of the present invention, the PCR sample contains primers. Although the nucleotide sequences of the primers are not particularly limited, the 2019-COVID primer sequences (N1) published by the Centers for Disease Control and Prevention (CDC) may be used as the primers. The sequences are available at http://www.cdc.gov/coronavirus/2019-ncov/downloads/rt-pcr-pane;-primer-probes.pdf, and any sequences may be used without limitation as long as they are for PCR.

According to one embodiment of the present invention, the PCR sample contains a probe. Although the nucleotide sequence of the probe is not particularly limited, the 2019-COVID probe sequence (N1) published by the Centers for Disease Control and Prevention (CDC) may be used as the probe. The is sequence available at http://www.cdc.gov/coronavirus/2019-ncov/downloads/rt-pcr-pane;-primer-probes.pdf, and any sequence may be used without limitation as long as it is for PCR.

According to one embodiment of the present invention, the PCR sample contains a premix. Although the premix is not particularly limited, Nanohelix RealHelix™ qRT-PCR Kit [v6] (UDG System) is preferably used as the premix, and any premix may be used without limitation as long as it is for PCR.

According to one embodiment of the present invention, the step of adding may comprise adding a PCR sample, containing a premix and a solution containing primers and a probe, to a well (or tube) containing a mixture containing a nucleic acid extracted by the method for cell lysis and nucleic acid extraction. As a sample is collected from the mixture and placed in a separate tube as described above and the PCR sample is not added, it is possible to minimize loss of the target gene by using the whole of the extracted nucleic acid for nucleic acid amplification, and to maximize PCR performance. In addition, it is possible to eliminate the need for a separate solution transfer process, thereby reducing PCR preparation time. Furthermore, it is possible to maximize the use of the nucleic acid exposed by thermal lysis of the cells and prevent the concentration from being diluted by the added PCR sample, thereby reducing the time required for molecular diagnosis and preventing PCR inhibition by the additives.

According to one embodiment of the present invention, the molecular diagnostic method includes a step (S190) of amplifying the extracted nucleic acid by polymerase chain reaction. Specifically, the step of amplifying by polymerase chain reaction may include sequentially performing RT-PCR (S191) and PCR (S193). By amplifying the extracted nucleic acid by polymerase chain reaction, it is possible to secure the nucleic acid for molecular diagnosis and minimize the time required for molecular diagnosis.

According to one embodiment of the present invention, the molecular diagnostic method may include amplifying the nucleic acid by polymerase chain reaction without separate purification of the well (or tube) to which the premix and the solution containing the primers and the probe have been added. As the nucleic acid is amplified by polymerase chain reaction without performing separate purification of the well to which the PCR sample has been added, as described above, it is possible to minimize the time required for molecular diagnosis.

One embodiment of the present invention provides the use of a composition, including an RNase inhibitor and a buffer, for cell lysis and nucleic acid extraction.

One embodiment of the present invention provides a kit for cell lysis and nucleic acid extraction or for molecular diagnosis including a composition including an RNase inhibitor and a buffer.

One embodiment of the present invention provides the use of a composition, including an RNase inhibitor and a buffer, for the preparation of a kit for cell lysis and nucleic acid extraction or molecular diagnosis.

In one embodiment of the present invention, when the composition including the RNase inhibitor and the buffer is used, it is possible to minimize the time required for molecular diagnosis by nucleic acid amplification by omitting elution and purification processes for a solution containing lysed cells and performing polymerase chain reaction using the solution containing lysed cells. Thus, the composition may be used for cell lysis and nucleic acid extraction, for a kit for cell lysis and nucleic acid extraction or molecular diagnosis, or for the preparation of the kit

Regarding the use of the composition for cell lysis and nucleic acid extraction, the use of the composition for a kit, and the use for the preparation of the kit according to an embodiment of the present invention, the composition for cell lysis and nucleic acid extraction, the molecular diagnostic method, the RNase inhibitor, and the buffer are described above.

### MODE FOR INVENTION

Hereinafter, the present invention will be described in detail with reference to examples. However, the examples according to the present invention may be modified into various different forms, and the scope of the present invention is not interpreted as being limited to the examples described below. The examples in the present specification are provided to more completely explain the present invention to those skilled in the art.

### <Compounds Used in Examples 1 to 4 and Conditions for Performing PCR>

The samples collected in Examples 1 to 4 below correspond to samples collected using clinical swab and stored in virus transport media, and purified target RNA was used as an RNA sample that was additionally added.

Furthermore, the RNase inhibitor used in Examples 1 to 4 below was an RNase inhibitor (Nanohelix RNase Inhibitor) derived from murine lung, and the buffer used in Examples 1 to 4 was a mixture of glycerol, hydroxyethyl piperazine ethane sulfonic acid (HEPES), dithiothreitol (DTT), and potassium chloride.

In addition, in Examples 1 to 4 below, as a probe in a PCR sample added to perform PCR, the 2019-COVID probe sequence (N1) published by the Centers for Disease Control and Prevention (CDC) was used, which is available at http://www.cdc.gov/coronavirus/2019-ncov/downloads/rt-pcr-pane;-primer-probes.pdf. As primes in the PCR sample, the 2019-COVID primer sequences (N1) published by the Centers for Disease Control and Prevention (CDC) were used, which are available at http://www.cdc.gov/coronavirus/2019-ncov/downloads/rt-pcr-pane;-primer-probes.pdf. Furthermore, as a premix in the PCR sample, the Nanohelix RealHelix™ qRT-PCR Kit [v6] (UDG System) was used.

In Examples 1 to 4 below, RT-PCR and PCR were performed under the following conditions: (1) 10 min at 50° C., (2) 5 min at 95° C., and then (3) 40 cycles, each consisting of 10 sec at 95° C. and 30 sec at 58° C. In this process, the threshold cycle (Ct) value that is the minimum threshold value capable of determining the result of nucleic acid amplification was measured.

### Example 1 (Evaluation of Effect of Thermal Lysis)

FIG. 4 shows a schematic diagram of molecular diagnostic methods according to Example 1 and a graph showing the Ct values measured in Examples 1-1 and 1-2.

Specifically, FIG. 4(a) is a schematic diagram of the molecular diagnosis methods according to Example 1. Referring to FIG. 4(a), in Example 1-1, a sample containing nucleic acid was collected, distilled water was added to the collected sample, and thermal lysis was performed at 95° C. for 5 minutes. Thereafter, a separately incubated RNA sample was added before performing RT-PCR, and then RT-PCR and PCR were performed sequentially.

Example 1-2 was performed in the same manner as Example 1-1, except that thermal lysis in Example 1-1 was not performed.

FIG. 4(b) is a graph showing the Ct values measured in Examples 1-1 and 1-2. Referring to FIG. 4(b), it was found that the Ct value in Example 1-1 was 0.5 lower than that in Example 1-2 because PCR inhibitory substances (RNases excluding RNase A) accompanying the sample were inactivated during the thermal lysis process.

### Example 2 (Evaluation of Effects of Different Types of RNase Inhibitors)

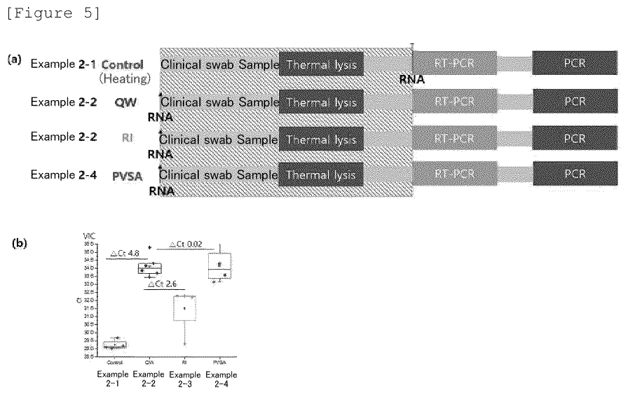

FIG. 5 shows a schematic diagram of molecular diagnostic methods according to Example 2 and a graph showing the Ct values measured in Examples 2-1 to 2-4.

Specifically, FIG. 5(a) is a schematic diagram showing the molecular diagnosis methods according to Example 2. Referring to FIG. 5(a), in Example 2-1, a sample containing nucleic acid was collected, distilled water was added to the collected sample, and thermal lysis was performed at 95° C. for 5 minutes. Thereafter, a separately incubated RNA sample was added before performing RT-PCR, and then RT-PCR and PCR were performed sequentially.

Example 2-2 was performed in the same manner as Example 2-1, except that the RNA sample was added to distilled water together with the collected sample, unlike Example 2-1.

Example 2-3 was performed in the same manner as Example 2-2, except that distilled water to which the RNase inhibitor was added was used instead of distilled water used in Example 2-2.

Example 2-4 was performed in the same manner as Example 2-2, except that distilled water to which PVSA (polyvinylsulfonic acid), an RNase inhibitor derived from the chemical substance, was added was used instead of distilled water used in Example 2-2.

FIG. 5(b) is a graph showing the Ct values measured in Examples 2-1 to 2-4. Referring to FIG. 5(b), it was found that, in Example 2-1, the Ct value was low due to amplification of the RNA sample added immediately before RT-PCR, because most of the RNases contained in the sample were inactivated by heat dissolution and only the remaining RNase A had some effect. In contrast, it was found that, in Example 2-2, the Ct value increased because the RNases contained in the sample has some effect before thermal lysis, and even when only the sequence was changed while maintaining the same conditions as those in Example 2-1, the Ct value was about 4.8 to 8 higher than that in Example 2-1. Furthermore, it was found that, in Example 2-3, the Ct value was about 2.6 lower than that in Example 2-2 because the RNase A was inactivated by addition of the RNase inhibitor together with the sample. However, it was found that, in Example 2-4, the Ct value was 0.02 higher than that in Example 2-2 due to the PCR inhibitory effect because the chemical-derived RNase inhibitor rather than a protein-derived RNase inhibitor was used.

### Example 3 (Evaluation of Effect of Buffer)

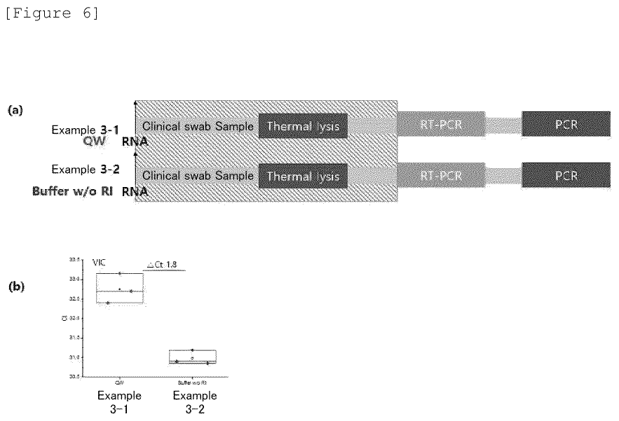

FIG. 6 shows a schematic diagram of molecular diagnostic methods according to Example 3 and a graph showing the Ct values measured in Examples 3-1 and 3-2.

FIG. 6(a) is a schematic diagram of the molecular diagnosis methods according to Example 3. Referring to FIG. 6(a), in Example 3-1, a sample containing nucleic acid was collected, distilled water and a separately incubated RNA sample were added to the collected sample, and thermal lysis was performed at 95° C. for 5 minutes. Thereafter, RT-PCR and PCR were performed sequentially.

Example 3-2 was performed in the same manner as Example 3-1, except that a buffer was used instead of distilled water used in Example 3-1.

FIG. 6(b) is a graph showing the Ct values measured in Examples 3-1 and 3-2. Referring to FIG. 6(b), it was found that the Ct value in Example 3-2 was 1.8 lower than that in Example 3-1, indicating that the PCR amplification effect was improved even when only the buffer was changed.

### Example 4 (Evaluation of Effects of Thermal Lysis Buffer and Different Types of RNase Inhibitors)

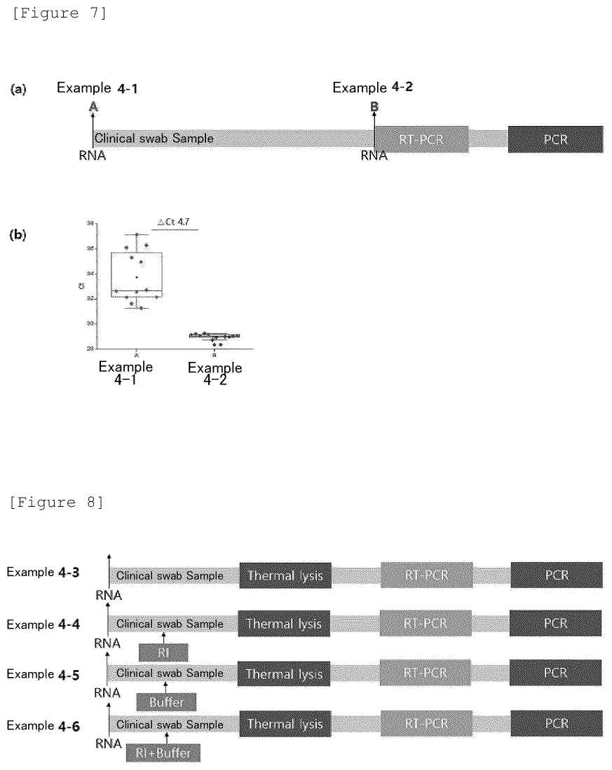

FIG. 7 shows a schematic diagram of molecular diagnostic methods according to Example 4 and a graph showing the Ct values measured in Examples 4-1 and 4-2.

Specifically, FIG. 7(a) is a schematic diagram of the molecular diagnosis methods according to Example 4. Referring to FIG. 7(a), in Example 4-1, a sample containing nucleic acid was collected, the collected sample was added to distilled water and a separately incubated RNA sample, and thermal lysis was performed at 95° C. for 5 minutes. Thereafter, RT-PCR and PCR were performed sequentially.

In Example 4-2, a sample containing nucleic acid was collected, the collected sample was added to distilled water, and thermal lysis was performed at 95° C. for 5 minutes. Thereafter, a separately incubated RNA sample was added before performing RT-PCR, and then RT-PCR and PCR were performed sequentially.

FIG. 7(b) is a graph showing the Ct values measured in Examples 4-1 and 4-2. Referring to FIG. 7(b), it could be found that, in Example 4-1, the Ct value was high because the RNA sample was degraded by the RNase present together with the collected sample, lowering the concentration of RNA for nucleic acid amplification. In contrast, it was found that, in Example 4-2, the Ct value was low at 4.7 due to a high concentration of the RNA, because the RNA sample was added immediately before RT-PCR for nucleic acid amplification and thus inactivation of a very small amount of the RNA occurred.

FIG. 8 is a schematic diagram showing molecular diagnostic methods according to Examples 4-3 to 4-6. Referring to FIG. 8, Example 4-3 was performed in the same manner as Example 4-1, except that the thermal lysis process was added to Example 4-1.

Example 4-4 was performed in the same manner as Example 4-3, except that an RNase inhibitor was added to the distilled water used in Example 4-3.

Example 4-5 was performed in the same manner as Example 4-3, except that a buffer was added to the distilled water used in Example 4-3.

Example 4-6 was performed in the same manner as Example 4-3, except that an RNase inhibitor and a buffer were added to the distilled water used in Example 4-3.

It was found that, in Example 4-3, the Ct value was 0.5 lower than that in Example 4-1, because the RNase contained in the collected sample was heat-inactivated during the thermal lysis process.

Furthermore, it was found that, in Example 4-4, the Ct value was 3.1 lower than that in Example 4-1, because the degradation of RNA was minimized due to the heat-inactivation effect confirmed in Example 4-3 and the removal of RNase A by the RNase inhibitor.

In addition, it was found that, in Example 4-5, the Ct value was 1.8 lower than that Example 4-1, indicating that the buffer improved the PCR amplification effect.

In addition, it was found that in Example 4-6, the Ct value was 4.9 lower than that in Example 4-1 and equivalent to that in Example 4-2, due to all of the heat inactivation effect confirmed in Example 4-3, the RNase A-inactivating effect of the RNase inhibitor as confirmed in Example 4-4, and because the buffer exhibited the effect of preventing PCR inhibitory factors as confirmed in Example 4-5.

### Preparation Example

In the Preparation Example below, a sample was collected using clinical swab and then stored in virus transport media.

Furthermore, the RNase inhibitor used in the Preparation Example was an RNase inhibitor (Nanohelix RNase Inhibitor) derived from murine lung, and the buffer used in the Preparation Example was a mixture of glycerol, hydroxyethyl piperazine ethane sulfonic acid (HEPES), dithiothreitol (DTT), and potassium chloride. In addition, a mixture was prepared by mixing the collected sample, the RNase inhibitor and the buffer.

In addition, in the Preparation Example below, as a probe in a PCR sample added to perform PCR, the 2019-COVID probe sequence (N1) published by the Centers for Disease Control and Prevention (CDC) was used, which is available at http://www.cdc.gov/coronavirus/2019-ncov/downloads/rt-pcr-pane;-primer-probes.pdf. As primes in the PCR sample, the 2019-COVID primer sequences (N1) published by the Centers for Disease Control and Prevention (CDC) were used, which are available at http://www.cdc.gov/coronavirus/2019-ncov/downloads/rt-pcr-pane;-primer-probes.pdf. Furthermore, as a premix in the PCR sample, the Nanohelix RealHelix™ qRT-PCR Kit [v6] (UDG System) was used.

In the Preparation Example below, RT-PCR and PCR were performed under the following conditions: (1) 10 min at 50° C., (2) 5 min at 95° C., and then (3) 40 cycles, each consisting of 10 sec at 95° C. and 30 sec at 58° C. In this process, the threshold cycle (Ct) value that is the minimum threshold value capable of determining the result of nucleic acid amplification was measured.

### Experimental Example 1 (Comparison with PCR Including Conventional Extraction Process)

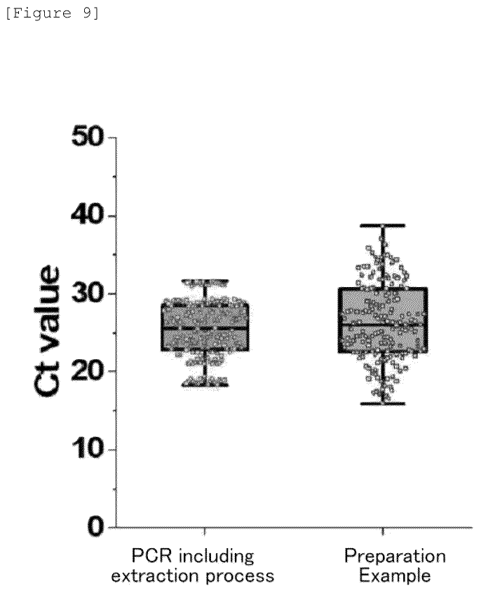

FIG. 9 is a graph showing the Ct value measured in a PCR including RNA extraction and purification processes according to a conventional art and the Ct value measured in the Preparation Example.

In the Preparation Example, a sample containing nucleic acid was collected, distilled water, an RNase inhibitor, and a buffer were added to the collected sample, and thermal lysis was performed at 95° C. for 5 minutes. Thereafter, RT-PCR and PCR were performed sequentially.

FIG. 9 shows a comparison between the result of performing PCR according to the conventional art shown in FIG. 1 and the result of the Preparation Example. It was found that, when the buffer containing the RNase inhibitor was added to the collected sample and thermal lysis was performed, the Ct value was realized at a level equivalent to that in the conventional PCR method.

### Experimental Example 2 (Evaluation of Effect Depending on Concentration of RNase Inhibitor in Mixture in First Heating Step)

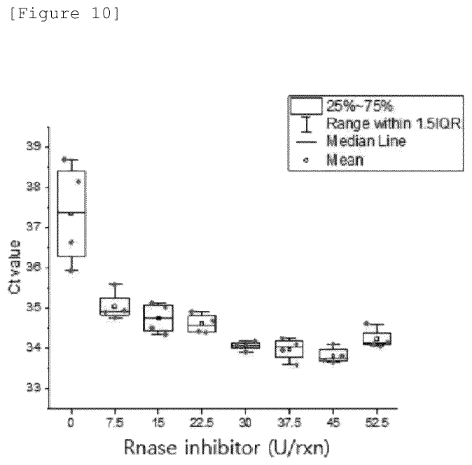

FIG. 10 is a graph showing a Ct value depending on the concentration of RNase inhibitor in the first heating (incubation) step of the Preparation Example.

Specifically, in the Preparation Example, the first heating step (incubation) was performed on the mixture at 37° C. for 5 minutes while changing the concentration of the RNase inhibitor in the mixture of the Preparation Example before performing RT-PCR and PCR, and the Ct value was measured depending on the concentration. More specifically, in the Preparation Example, a sample containing nucleic acid was collected, the collected sample was added to distilled water, an RNase inhibitor, and a buffer, and thermal lysis was performed at 95° C. for 5 minutes. Thereafter, while the concentration of the RNase inhibitor in the mixture of the Preparation Example was changed, the mixture was subjected to the first heating step (incubation) at 37° C. for 5 minutes, and then RT-PCR and PCR were performed sequentially. The changed concentrations were 0 units/reaction (U/rxn), 7.5 U/rxn, 15 U/rxn, 22.5 U/rxn, 30 U/rxn, 37.5 U/rxn, 45 U/rxn, and 52.5 U/rxn, and the Ct value was measured for each concentration.

Referring to FIG. 10, it was found that, at 0 U/rxn, the Ct value was high because no RNase inhibitor was included. Thereafter, it was found that, at a concentration of 7.5 U/rxn to 45 U/rxn, the Ct value gradually decreased as the concentration of the RNase inhibitor increased. However, it was found that, at 52.5 U/rxn, the Ct value increased even when the concentration of the RNase inhibitor increased, but the Ct value was lower than that at a concentration of 7.5 U/rxn.

### Experimental Example 3 (Evaluation of Effect Depending on Temperature of First Heating Step)

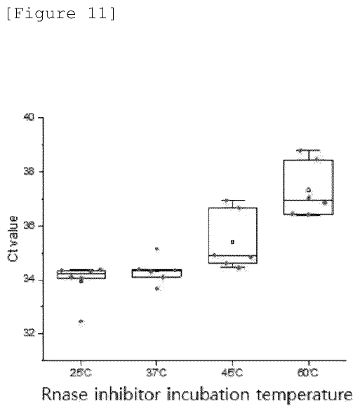

FIG. 11 is a graph showing a Ct value depending on the temperature of the first heating step of the Preparation Example.

Specifically, in the Preparation Example, before performing RT-PCR and PCR, the concentration of an RNase inhibitor in the mixture of the Preparation Example was fixed at 30 U/rxn and the mixture was subjected to the first heating step (incubation) for 5 minutes while changing the temperature, and the Ct value was measured depending on the temperature. More specifically, in the Preparation Example, a sample containing nucleic acid was collected, the collected sample was added to distilled water, an RNase inhibitor and a buffer, and thermal lysis was performed at 95° C. for 5 minutes. Thereafter, the concentration of the RNase inhibitor in the mixture of the Preparation Example was fixed at 30 U/rxn, and the first heating step (incubation) was performed for 5 minutes while changing the temperature of the mixture, and then RT-PCR and PCR were performed sequentially. The changed temperatures were 25° C., 37° C., 45° C., and 60° C., and the Ct value was measured for each temperature.

Referring to FIG. 11, it was found that the Ct value was constant at a temperature of 25° C. to 37° C. Thereafter, it was found that the Ct value increased at a temperature of 45° C. or higher, indicating that the RNase inhibitor was inhibited at a temperature of 45° C. or higher.

Therefore, the composition for cell lysis and nucleic acid extraction, the nucleic acid extraction method using the same, and the molecular diagnostic method using the same according to one embodiment of the present invention may inactivate RNase by heating while using the composition for nucleic acid extraction containing the RNase inhibitor, thereby omitting a separate nucleic acid purification process and shortening the overall experiment time, and improving PCR performance by minimizing damage to RNA.

Although the present invention has been described above with reference to limited embodiments, it should be understood that the present invention is not limited to these embodiments, and various modifications and variations may be made by those skilled in the art without departing from the technical idea of the present invention and within the range of equivalents to the following claims.

### INDUSTRIAL APPLICABILITY

The composition for cell lysis and nucleic acid extraction according to the present invention may minimize the time required for molecular diagnosis by nucleic acid amplification by omitting a separate elution process and purification process for a solution containing lysed cells and performing polymerase chain reaction using the solution containing lysed cells, and improve the accuracy of molecular diagnosis by inactivating factors, which impede the accuracy of polymerization chain reaction, through heating in the process of extracting nucleic acid. Thus, the composition is industrially applicable.

1. A composition for cell lysis and nucleic acid extraction comprising an RNase inhibitor and a buffer.

2. The composition according to claim 1, wherein RNase inhibitor comprises an RNase A inhibitor.

3. The composition according to claim 1, wherein RNase inhibitor is derived from protein.

4. The composition according to claim 1, wherein the buffer has a pH of 6.0 to 9.0.

5. The composition according to claim 1, wherein the buffer contains any one selected from among glycerol, hydroxyethyl piperazine ethane sulfonic acid (HEPES), dithiothreitol (DTT), potassium chloride, and combinations thereof.

6. A method for cell lysis and nucleic acid extraction comprising:
a step of preparing a mixture by adding a sample containing nucleic acid to the composition for cell lysis and nucleic acid extraction according to claim 1;
a first heating step of maintaining the mixture at a temperature of 25° C. to 45° C.; and
a second heating step of maintaining the mixture resulting from the first heating step at a temperature of 75° C. or higher to lower than 100° C.

7. The method according to claim 6, wherein the first heating step and the second heating step are each performed for 1 minute to 30 minutes.

8. The method according to claim 6, wherein a concentration of the RNase inhibitor in the mixture is 7.5 units/reaction to 60.0 units/reaction, with respect to the volume of the mixture being 30 μL.

9. A molecular diagnostic method comprising:
a step of adding a premix and a solution containing primers and a probe to a mixture containing a nucleic acid extracted by the method for cell lysis and nucleic acid extraction according to claim 6; and
amplifying the extracted nucleic acid by polymerase chain reaction.

10. (canceled)
